(19) United States
(12) Patent Application Publication
(10) Pub. No.: US 2005/0277693 A1
(43) Pub. Date: Dec. 15, 2005

(54) Basic salts and monohydrates of certain alpha, beta-propionic acid derivative

(75) Inventors: Venkata Raghavendra Charyulu Palle, Hyderabad (IN); Hari Charan Raju Chennamadhauni, Hyderabad (IN)

Correspondence Address: DR. REDDY'S LABORATORIES, INC., 200 SOMERSET CORPORATE BLVD, SEVENTH FLOOR,, BRIDGEWATER, NJ, 08807-2862, US

(73) Assignees: DR. REDDY'S LABORATORIES LIMITED; DR. REDDY'S LABORATORIES, INC.

(21) Appl. No.: 10/865,508

(22) Filed: Jun. 10, 2004

(51) Int. Cl. A61K 31/205

(52) U.S. Cl. 514/554; 558/48

(57) ABSTRACT

The invention provides novel salts of (S)-2-methoxy-3-[4-{3-(4-methanesulfonyloxyphenyl)propylamino} phenyl] propionic acid, including Arginine salt of (S)-2-methoxy-3-[4-{3-(4-methanesulfonyloxyphenyl)propylamino} phenyl] propionic acid monohydrate, Arginine salt of (S)-2-methoxy-3-[4-{3-(4-methanesulfonyloxyphenyl) propylamino} phenyl] propionic acid, and (S)-2-methoxy-3-[4-{3-(4-methanesulfonyloxyphenyl)propylamino} phenyl] propionic acid monohydrate. Various aspects and embodiments are included. Compositions containing novel salts of (S)-2-methoxy-3-[4-{3-(4-methanesulfonyloxyphenyl) propylamino}phenyl] propionic acid are also provided.

### BACKGROUND OF THE INVENTION

Formulation of a convenient pharmaceutical dosage form can be very complicated. While there are many factors that contribute to the design criteria, perhaps the most important is the active pharmaceutical ingredient (“API”) that will be delivered. The API's solubility, route of administration, dosage size, taste, absorption target or cite of application, metabolic properties and the like often must all be considered. And if the API has stability and/or handling issues, the complexity rises accordingly. Therefore, where possible, it is highly desirable to develop APIs that can be conveniently handled and processed. Chemical stability, solid-state stability and shelf life of the active ingredients are also important considerations. To the extent that the API is stable, non-reactive with common excipients under normal processing and storage conditions and the like, dosage form development can be greatly facilitated.

The API and compositions containing it should be capable of being effectively stored over appreciable periods of time, without exhibiting a significant change in the physico-chemical characteristics of active compounds. Crystalline materials, for example, can in certain cases be less difficult to handle and to formulate when compared to amorphous forms. But stable crystalline forms that are suitable for formulation and provide sufficient solubility and bioavailabilty are neither necessarily available nor predictable, especially when complex molecules are involved. Moreover, some crystalline materials are not sufficiently stable to ensure that they will not convert to another form, crystalline or not, during manufacturing or storage.

Salt formation may improve certain properties such as stability, water solubility and bioavailability. Salts may also influence hygroscopicity and crystallinity. Of course, there is a vast array of possible salts (See WO 03/048116 at 21, line 18-22, line 4) not all salts can be made are equally easy to make or equally advantageous. And not all salts permit the same crystalline forms. The nature of the salts can also influence can the properties of the API and the ability to make a stable dosage form using modern, high speed equipment in an efficient and cost effective manner. And, perhaps most importantly, there must be no unnecessary impediment to the safety and efficacy of the API.

### SUMMARY OF THE INVENTION

The present invention includes the basic salts of ((S)-2-methoxy-3-[4-{3-(4-methanesulfonyloxyphenyl) propylamino}phenyl]propionic acid) (also referred to herein as the basic salts of “(S)MP”) and, in particular, the amino acid salts thereof. A particularly preferred amino acid salt in accordance with the present invention is the arginine salt of (S)MP (the “Arginine salt” or “(S)MP-Arg”.). The invention also encompasses these salts in pure or substantially pure forms.

The present invention also includes the monohydrate form of (S)MP as well as the monohydrate of the basic salts of (S)MP, preferably the monohydrate of the amino acid salts of (S)MP, and most preferably (S)MP arginine monohydrate (also referenced as “Arginine salt monohydrate” or “(S)MP-Arg.1H₂O”). The invention also encompasses these monohydrates in a pure or substantially pure forms.

Another aspect of the present invention is crystalline forms of the basic salts of (S)MP, whether in monohydrated form or not.

A particularly preferred embodiment of the present invention is a crystalline form of (S)MP monohydrate or (S)MP-Arg.1H₂O.

Another aspect of the present invention provides one or more of the foregoing salts and/or monohydrates of (S)MP as the API in a novel pharmaceutical product which is stable and can be efficiently manufactured using traditional high speed equipment. These pharmaceutical products include one of the foregoing as the API salts and/or monohydrates of (S)MP and a pharmaceutically acceptable carrier, diluent, excipient or solvent.

Another particularly preferred aspect of the present invention is a pharmaceutical composition that contains, as the API, one or more of the forgoing salts and/or monohydrates of (S)MP and, in particular an Arginine salt and/or Arginine salt monohydrate, in crystalline form, wherein the API is present in an amount of at least about 0.01% by weight of the pharmaceutical composition.

Another aspect of the present invention provides a process for the preparation of one or more of the foregoing salts and/or monohydrates of (S)MP and, in particular an (S)MP monohydrate Arginine salt and/or Arginine salt monohydrate, in a crystalline form.

### DESCRIPTION OF THE DRAWINGS

FIG. 1 is an infrared spectrum of one batch of Arginine monohydrate of ((S)-2-methoxy-3-[4-{3-(4-methanesulfonyloxyphenyl) propylamino}phenyl]propionic acid).

FIG. 2 is an X-ray powder diffractogram of one batch of Arginine monohydrate of ((S)-2-methoxy-3-[4-{3-(4-methanesulfonyloxyphenyl) propylamino}phenyl]propionic acid).

FIG. 3 is a DSC thermogram of one batch of Arginine monohydrate of ((S)-2-methoxy-3-[4-{3-(4-methanesulfonyloxyphenyl) propylamino}phenyl]propionic acid).

FIG. 4 is an infrared spectrum of one batch of Arginine salt of ((S)-2-methoxy-3-[4-{3-(4-methanesulfonyloxyphenyl) propylamino}phenyl]propionic acid).

FIG. 5 is an X-ray powder diffractogram of one batch of Arginine salt of ((S)-2-methoxy-3-[4-{3-(4-methanesulfonyloxyphenyl) propylamino}phenyl]propionic acid).

FIG. 6 is a DSC thermogram of one batch of Arginine salt of ((S)-2-methoxy-3-[4-{3-(4-methanesulfonyloxyphenyl) propylamino}phenyl]propionic acid).

### DETAILED DESCRIPTION

A compound (also referred to herein as a molecule or chemical) that may be used as a starting material for the preparation of salts and hydrates of the present invention, can be prepared according to known procedures, such as those disclosed in, inter alia, International Publication No. WO 03/048116, which is incorporated herein by reference both in its entirety and for the purpose stated. See in particular Example 14.

As used herein, the salts and monohydrates of the present invention refer to a molecule, per se, unless expressly stated otherwise or as is appropriate under the circumstances. When reference is made to a crystal or crystalline material, a sufficient number of molecules necessary to make a single crystal is contemplated. In the context of a pharmaceutical dosage form, pharmaceutical product or pharmaceutical composition, at least 0.10% by weight of the product is a basic salt of (S)MP or a monohydrate thereof as disclosed herein.

The present invention provides basic salts of (S)MP, including those in crystalline form. Examples of basic salts include salts of alkali and earth alkali metals, and amino acid salts (both naturally occurring and synthetic amino acids). Preferably, the salt is an arginine salt of compound (I) (“Arginine salt”), which was found to be particularly suitable.

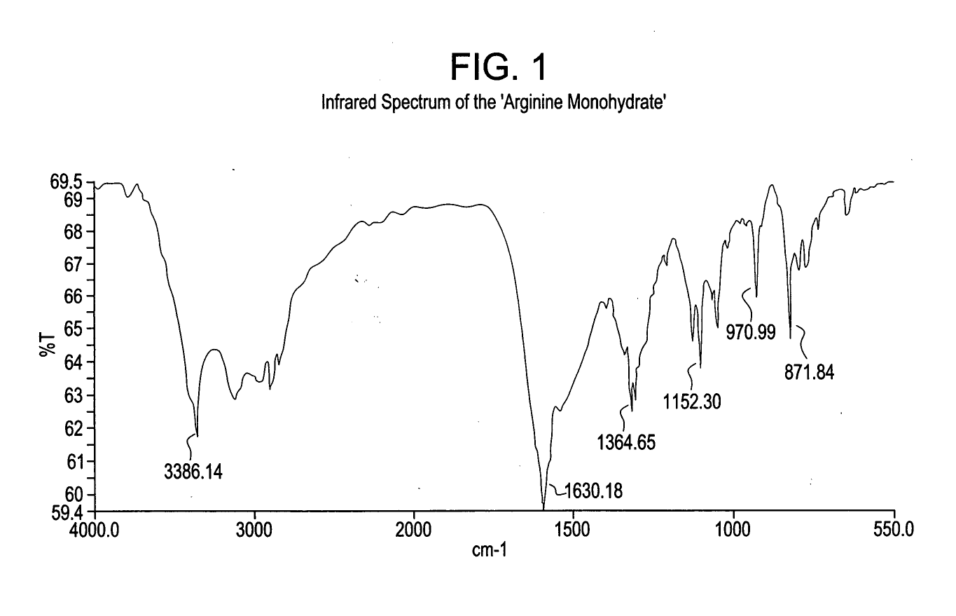

The present invention encompasses monohydrates of (S)MP and its basic salts as novel materials. Monohydrates ideally include a ratio of one molecule of (S)MP or its basic salt with one molecule of bound water of crystallization. Preferred are monohydrates of basic salts and more preferably Arginine salts of (S)MP. Most preferred are (S)MP monohydrate and Arginine salt monohydrate. Even more preferred is the Arginine salt monohydrate having the characteristics of at least one of FIGS. 1-3.

The present invention also provides a process for preparing basic salts and monohydrates of (S)MP and in particular amino acid salt monohydrates thereof. In particular, Arginine monohydrate of (S)MP or a salt thereof is preferably dispersed or dissolved in a suitable solvent, and is reacted with a suitable source of arginine ion in the presence of water, which comprises:
(i) refluxing (S)MP, DM water and Arginine′;
(ii) further refluxing with additional quantities of a suitable reaction solvent, preferably alcoholic solvent, more preferably, isopropanol;
(iii) cooling and filtering the reaction mixture to obtain the Arginine salt hydrate.

The concentration of (S)MP is preferably in the range of about 60 to 70% weight/weight, more preferably in the range of about 65 to 69%. The concentration of arginine is preferably in the range of about 21 to 39% weight/weight, more preferably in the range of about 26 to 33%. The concentration of water is in the range of about 1.0 to 9.0%.

Formation of a monohydrate requires the presence of water at some stage; water may be added as a co-solvent in the above process, e.g., about 5 to 100% water. However, it is also possible to provide sufficient water for monohydrate formation by carrying out the reaction with exposure to atmospheric moisture, or by use of non-anhydrous solvents.

A suitable reaction solvent for the preparation of arginine salt monohydrate is a ketone, such as acetone, ethyl methyl ketone, ether such as tetrahydrofuran, dioxane, isopropyl ether, diethyl ether, an alcohol such as methanol, ethanol, propan-2-ol, isopropanol or mixtures thereof and the like. The temperature used in the reaction is maintained in the range of about 30 to 120° C., preferably about 30 to 70° C. The duration of the reaction is maintained in the range of about 3 to 12 h, preferably, for approximately 5 hours.

In one aspect of this invention, the Arginine salt monohydrate has been reproducibly isolated, containing water ranging from about 2.6-3.4% by weight/weight, preferably from about 2.9-3.4% by weight/weight, preferably from about 3.0% by weight/weight. This is consistent with a monohydrate formation (1:1 ratio of Arginine salt of compound (I) and water) (1 molar equivalent of H₂O=3.00% by weight/weight) has been isolated.

The crystalline form may be characterized by X-ray powder diffraction pattern (XRPD). X-ray powder diffraction pattern analysis was performed on Rigaku D/Maz-2200 model diffractometer equipped with horizontal goniometer in θ/2θ geometry. The Copper Kα(λ=1.5418 Å) radiation was used. The tube voltage and amperage were set at 50 KV and 34 mA respectively. The divergence and scattering slits are set at ½ degree, receiving slit at 015 mm and scattering slit at ½ degree. Diffracted radiation is detected by scintillation counter detector, θ to 2θ continuous scan at 3 degrees/minutes from 3 to 45 degrees be used. A standard was analyzed to check the instrumental alignment the data was collected and analyzed using.

Differential scanning calorimetry was performed using a Shimadzu DSC-50 calorimeter. The sample was placed into aluminum pan, the weight was accurately recorded, and the pan was covered with lid and left unclamped. Each sample was equilibrated and heated (10° C./minute) under nitrogen atmosphere.

The crystalline form may also characterized by Fourier transform infra red spectra and recorded in solid state as KBr dispersion. Perkin-Elmer 1600 Fourier transform infra red spectrophotometer was used for characterization.

In one suitable embodiment, there is provided an Arginine salt monohydrate of (S)MP (1 molar equivalent of H₂O=3.00% by weight/weight), characterized by one or more of the following:
(i) Infra red spectrum containing peaks at about 1630, 1364, 1152, 971, 871 cm⁻¹;
(ii) X-Ray powder diffraction (XRPD) pattern containing peaks at about 8.44, 17.48, 17.60, 18.84, 21.02, 21.18, 22.02 degrees 2θ; and/or
(iii) Endotherms at about 92.60, 132.53, 231.80 and 272.22° C. in Differential Scanning Calorimeter (DSC).

In one preferred aspect, Arginine monohydrate has infrared spectrum containing peaks at 1630, 1364, 1152, 971, 871 cm⁻¹. Preferably, the infrared spectrum is substantially in accordance with FIG. 1.

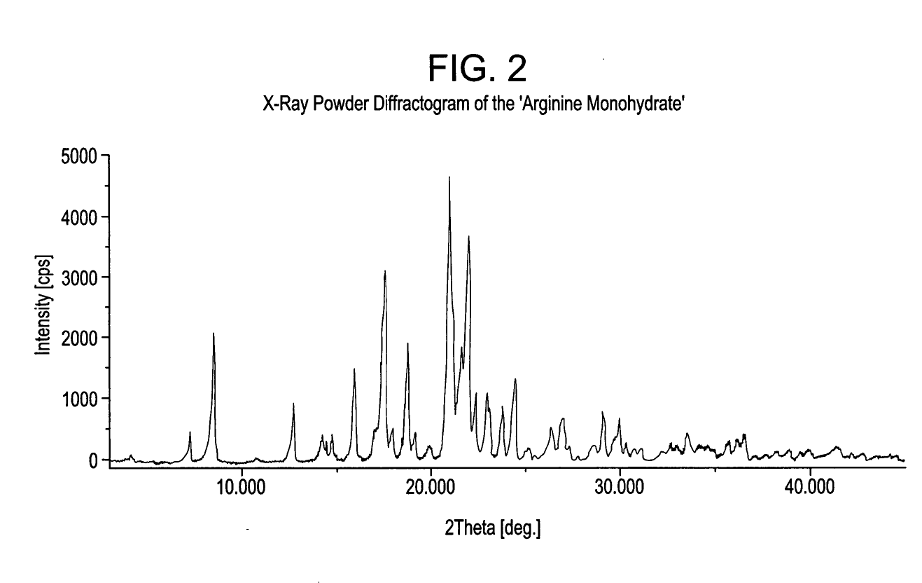

In one preferred aspect, an Arginine monohydrate provides X-Ray powder diffraction (XRPD) pattern substantially as shown in FIG. 2, and preferably has peaks at 8.44, 17.48, 17.60, 18.84, 21.02, 21.18, 22.02 degrees 2θ±0.2°2θ.

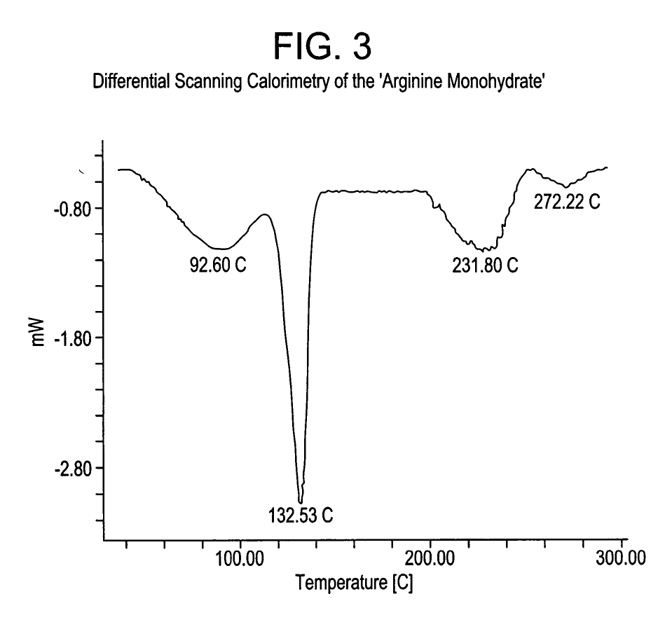

In one preferred aspect, an Arginine monohydrate provides a Differential Scanning Calorimeter (DSC) spectrum substantially as shown in accordance with FIG. 3, and preferably has endotherms at about 92.60, 132.53, 231.80 and 272.22° C.

It is to be understood that the X-ray diffraction (XRD) patterns, IR spectra and endotherms reported herein, while reported as absolute numbers in tables and absolute positions in the figures, are intended to include the normal amount of positional variation due to experimental error, operator error, differences in equipment, technique, packing, contamination and the like. It is understood by one skilled in the art that there may be substantial variation in the measured values. For example, it is believed the measurement of XRD peaks may have variability of ±0.2°2θ. However, based on these techniques, particularly when two or more of them are used in conjunction, and the overall spectrum and/or patterns reported in the figures, one of ordinary skill in this art will be able to identify whether or not a compound is a basic salt or basic salt monohydrate of (S)MP in accordance with the invention.

It should be kept in mind that slight variations in the observed 2 theta angles values are to be expected based on the specific diffractometer employed, the analyst and the sample preparation technique. More variation is expected for the relative peak intensities, which is largely affected by the particle size of the sample. Thus, identification of the exact crystalline form of a compound should be based primarily on observed 2 theta angles with lesser importance attributed to relative peak intensities. The peaks reported herein are listed in order of their peak intensities. Thus, the first listed peak has stronger intensity than the second listed peak in the pattern. For example, the 2 theta diffraction angles and corresponding d-spacing values account for positions of various peaks in the X-ray powder diffraction pattern. D-spacing values are calculated with observed 2 theta angles and wavelength using the Bragg equation well known to those of skill in the art.

In a further preferred aspect of the invention, an Arginine salt monohydrate has a melting point in the range of 125 to 140° C., such as 127 to 135° C., for example 132.5° C.

In a further preferred aspect of the invention, the invention provides Arginine salt monohydrate, characterized in that it provides at least one of an:
(i) Infrared spectrum substantially in accordance with FIG. 1;
(ii) X-Ray powder diffraction (XRPD) pattern substantially in accordance with Table 1 or FIG. 2; and/or
(iii) Endotherm at about 132.5° C. in Differential Scanning Calorimeter (DSC) spectrum substantially in accordance with FIG. 3.

The arginine salt of (S)MP can be produced by reacting (S)MP or some other salt thereof, preferably dispersed or dissolved in a suitable solvent, is reacted with a suitable source of arginine ion in the presence of a solvent in the 1:1 ratio of (S)MP and arginine, which comprises:
(i) refluxing compound (I), DM water and arginine
(ii) further refluxing with suitable reaction solvent,
(iii) azeotropically separating the product obtained by using suitable solvent cooling and filtering the resultant product to obtain arginine salt of (S)MP.

The concentration of (S)MP is preferably in the range of about 60 to 80% weight/weight, more preferably in the range of about 65 to 75%. The concentration of arginine is preferably in the range of about 20 to 40% weight/weight, more preferably in the range of about 25 to 35%.

A suitable reaction solvent for the preparation of anhydrous form is a ketone, such as acetone, ethyl methyl ketone, ether such as tetrahydrofuran, dioxane, diisopropyl ether, diethylether, an alcohol, such as methanol, ethanol, propan-2-ol. The solvent used during the azeotropic removal of water is selected from a hydrocarbon such as xylene, toluene, an ester such as ethyl acetate, a nitrile such as acetonitrile, or a halogenated hydrocarbon such as dichloromethane, dichloroethane and the like. The temperature used in the reaction is maintained in the range of about 30 to 120° C., preferably about 30 to 70° C. The duration of the reaction is maintained in the range of about 3 to 12 h.

Accordingly, the present invention provides arginine salt of (S)MP in nonhydrated form ((S)MP-Arg) as a novel compound, which is characterized by at least one of an:
(i) Infra red spectrum containing peaks at 1638, 1364, 1173, 1152, 971, 871 cm⁻¹;
(ii) X-Ray powder diffraction (XRPD) pattern containing peaks at 16.40, 16.82, 17.38, 19.28, 19.70, 20.22, 20.56 degrees 2θ; and/or
(iii) Endotherm at about 192.9° C. and 228.7° C.

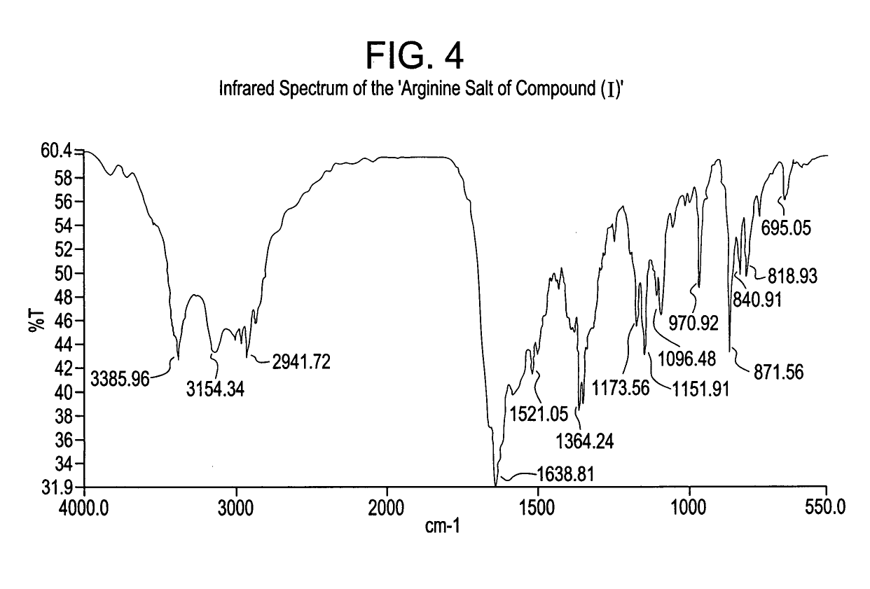

In one preferred aspect, the (S)MP-Arg provides an infrared spectrum substantially in accordance with FIG. 4.

In one preferred aspect, (S)MP-Argis characterized by an infrared spectrum containing peaks at about 1638, 1364, 1173, 1152, 971, 871 cm⁻¹.

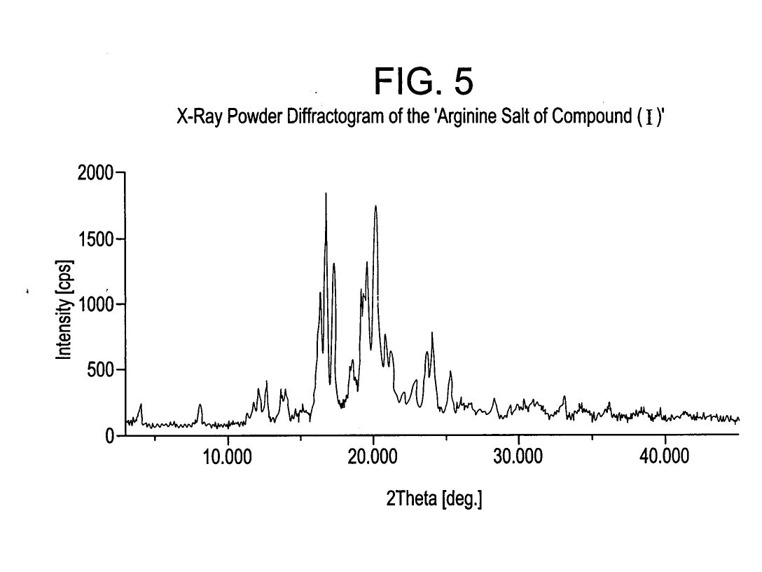

In one preferred aspect, the (S)MP-Arg provides X-Ray powder diffraction (XRPD) substantially in accordance with Table 2 or FIG. 5.

In one preferred aspect, the (S)MP-Arg characterized by an X-ray powder diffraction (XRPD) pattern containing peaks at about 16.40, 16.82, 17.38, 19.28, 19.70, 20.22, 20.56 degrees 2θ±0.2°2θ.

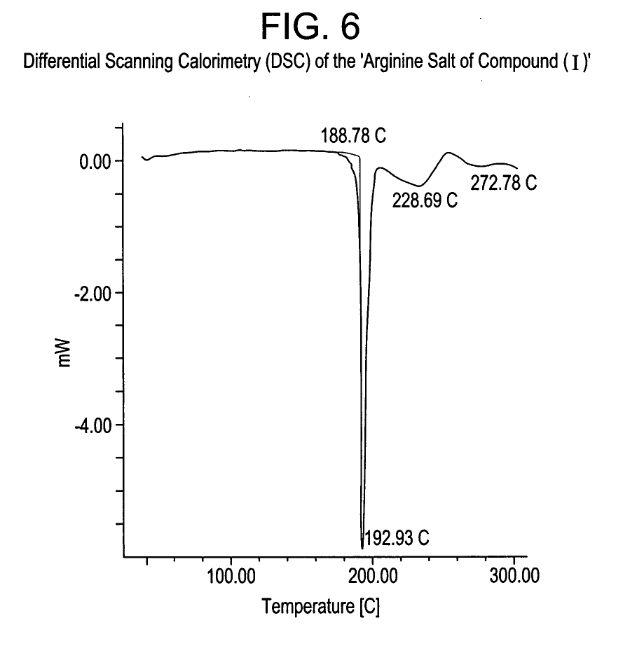

In one preferred aspect, the (S)MP-Arg provides a Differential Scanning Calorimeter (DSC) spectrum substantially in accordance with FIG. 6.

In one preferred aspect, the (S)MP-Arg is characterized by a Differential Scanning Calorimeter (DSC) spectrum containing endotherm at about 192.9 and 228.7° C.

In a further preferred aspect the (S)MP-Arg has a melting point in the range of about 185 to 200° C., such as 187 to 195° C., for example, 192.9° C.

In a further preferred aspect, the invention provides a (S)MP-Arg characterized in that it provides at least one of an:
(i) Infrared spectrum substantially in accordance with FIG. 4;
(ii) X-Ray powder diffraction (XRPD) pattern substantially in accordance with FIG. 5; and/or
(iii) Endotherm at about 192.9° C. in Differential Scanning Calorimeter (DSC) spectrum substantially in accordance with FIG. 6.

The invention also provides a process for the conversion of the Arginine salt monohydrate to (S)MP-Arg, wherein the Arginine salt monohydrate is suspended in a suitable solvent and refluxed to remove water azeotropically, which comprises:
(i) refluxing Arginine salt monohydrate with a suitable solvent to azeotropically remove water content,
(ii) cooling and filtering the resultant product to obtain Arginine salt of compound (I).

The solvent used during the azeotropic removal of water is selected from a hydrocarbon such as xylene, toluene, an ester such as ethyl acetate, a nitrile such as acetonitrile, or a halogenated hydrocarbon such as dichloromethane, dichloroethane and the like. The temperature used in the reaction is maintained in the range of about 30 to 80° C., preferably about 30 to 70° C. The duration of the reaction is maintained in the range of about 3 to 12 h.

The invention also provides a process for the conversion of the (S)MP-Arg to Arginine monohydrate, wherein the (S)MP-Argis suspended in a suitable solvent in the presence of water, and optionally thereafter as required:
(i) refluxing (S)MP-Arg and DM water,
(ii) further refluxing with suitable reaction solvent,
(iii) cooling and filtering the reaction mixture to obtain the Arginine monohydrate.

A suitable reaction solvent for the preparation of Arginine monohydrate is a ketone, such as acetone, ethyl methyl ketone an ether such as tetrahydrofuran, dioxane, isopropyl ether, diethyl ether, an alcohol such as methanol, ethanol, propan-2-ol, isopropanol or mixtures thereof and the like. The temperature used in the reaction is maintained in the range of about 30 to 80° C., preferably about 30 to 70° C. The duration of the reaction is maintained in the range of about 3 to 12 h.

Recovery of the desired compound may comprise crystallization from a solvent, conveniently the reaction solvent, usually assisted by cooling. For example, from an alkanol such as propan-2-ol or a hydrocarbon such as toluene. An improved yield of the badic salts may be obtained by evaporation of some or all of the solvent or by crystallization at elevated temperature, for example about 75° C., followed by slow cooling. Co-solvents can be added to reduce the solubility of the product in the solvent system to provide a good yield, e.g. diethyl ether, diisopropyl ether and heptane. Careful control of precipitation temperature and seeding may be used to improve the reproducibility of the product form.

Crystallization may also be initiated by, in one non-limiting example, seeding.

When used herein the term “Tₒₙₛₑₜ” is generally determined by Differential Scanning Calorimetry and has a meaning generally understood in the art, as for example expressed in “Pharmaceutical Thermal Analysis, Techniques and Applications”, Ford and Timmins, 1989 as “The temperature corresponding to the intersection of the pre-transition baseline with the extrapolated leading edge of the transition”.

“Substantially pure” means the material that contains about 95-99.8%, at least about 95%, preferably about 98%, more preferably about 99.8% of the compounds of the present invention. “Pure” means greater than 99.8% of the compound is the desired compound. This is, of course, not counting excipients, diluents, carriers or solvents and other pharmaceutically active molecules in the case of a pharmaceutical composition.

As mentioned above the compounds of the invention have useful therapeutic properties of the type: The present invention accordingly provides (S)MP in monohydrated form, as a basic salt or as a basic salt monohydrate for use as an active therapeutic substance disclosed for other members of the claim described in WO 03/048116. These molecules form may be administered per se or, preferably, as a pharmaceutical composition also comprising a pharmaceutically acceptable carrier, diluent or excipient.

Accordingly, the present invention also provides pharmaceutical compositions comprising the basic salts of (S)MP, (S)MP monohydrate and monohydrated forms of basic salts of (S)MP, and more preferably (S)MP-Arg and (S)MP-1H2O mixed with, solubilized or dispersed in a pharmaceutically acceptable carrier, diluent, solvent and/or the excipient. Preferably, the basic salt of (S)MP, (S)MP monohydrate or monohydrated form of a basic salt of (S)MP (collectively the “API”) is in a crystalline form. When present in such pharmaceutical compositions of pharmaceutical products, the API should be present in an amount of at least about 0.10% by weight of the pharmaceutical product. When the API is in crystalline form, at least one crystal should be present, although preferably at least about 0.10% by weight should be present.

These APIs and in particular, Arginine salt monohydrate or Arginine salt are normally administered in unit dosage form.

The API may be administered by any suitable route but usually by the oral or parenteral routes. For such use, the compound will normally be employed in the form of a pharmaceutical composition in association with a pharmaceutical carrier, diluent and/or excipient, although the exact form of the composition will naturally depend on the mode of administration.

The pharmaceutical compositions are prepared by admixture and are suitably adapted for oral, parenteral or topical administration, and as such may be in the form of tablets, capsules, oral liquid preparations, powders, granules, lozenges, pastilles, reconstitutable powders, injectable and infusible solutions or suspensions, suppositories and transdermal devices. Orally administrable compositions are preferred.

Tablets and capsules for oral administration are usually presented in a unit dose, and contain conventional excipients such as binding agents, fillers, diluents, tableting agents, lubricants, disintegrants, colorants, flavorings, and wetting agents. The tablets may be coated according to methods known in the art.

Suitable fillers for use include cellulose, mannitol, lactose and other similar agents. Suitable disintegrants include starch, polyvinylpyrrolidone and starch derivatives such as sodium starch glycolate. Suitable lubricants include, for example, magnesium stearate. Suitable pharmaceutically acceptable wetting agents include sodium lauryl toluenesulfonate.

Solid oral compositions may be prepared by conventional methods of blending, filling, tableting or the like. Repeated blending operations may be used to distribute the active agent throughout those compositions employing large quantities of fillers. Such operations are, of course, conventional in the art.

Oral liquid preparations may be in the form of, for example, aqueous or oily suspensions, solutions, emulsions, syrups, or elixirs, or may be presented as a dry product for reconstitution with water or other suitable vehicle before use. Such liquid preparations may contain conventional additives such as suspending agents, for example sorbitol, syrup, methyl cellulose, gelatin, hydroxyethylcellulose, carboxymethyl cellulose, aluminum stearate gel or hydrogenated edible fats, emulsifying agents, for example lecithin, sorbitan monooleate, or acacia; non-aqueous vehicles (which may include edible oils), for example, almond oil, fractionated coconut oil, oily esters such as esters of glycerine, propylene glycol, or ethyl alcohol; preservatives, for example methyl or propyl p-hydroxybenzoate or sorbic acid, and if desired conventional flavoring or coloring agents.

For parenteral administration, fluid unit dose forms are prepared containing a compound of the present invention and a sterile vehicle. The compound, depending on the vehicle and the concentration, can be either suspended or dissolved. Parenteral solutions are normally prepared by dissolving the active compound in a vehicle and filter sterilizing before filling into a suitable vial or ampoule and sealing. Advantageously, adjuvants such as a local anesthetic, preservatives and buffering agents are also dissolved in the vehicle. To enhance the stability, the composition can be frozen after filling into the vial and the water removed under vacuum.

Parenteral suspensions are prepared in substantially the same manner except that the active compound is suspended in the vehicle instead of being dissolved and sterilized by exposure to ethylene oxide before suspending in the sterile vehicle. Advantageously, a surfactant or wetting agent is included in the composition to facilitate uniform distribution of the active compound.

As is common practice, the compositions will usually be accompanied by written or printed directions for use in the medical treatment concerned. The unit dose compositions of the invention comprise the API in an amount providing up to about 12 mg, including about 0.1-12 mg.

The invention is illustrated by examples which are not intended to limit the invention in any way.

### EXAMPLE 1

### Preparation of [(S) 3-Methoxy-3-[4-{3-(4-methanesulfonyoxyphenyl) propylamino}phenyl]propionic acid] ((S)MP)

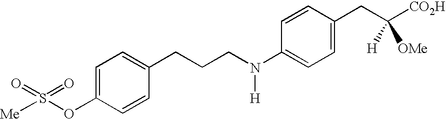

Step (i)

To a suspension of LAH (22.1 g, 2.5 eq, 583 mmol) in dry THF (1.0 L), was added dropwise a THF (50 mL) solution of methyl 3-(4-hydroxyphenyl)propionate (21 g, 1.0 eq, 116 mmol) at RT. The reaction mixture was refluxed for 4-5 h. It was worked up by quenching with excess ethyl acetate followed by addition of water (23 mL), 15% aq. NaOH (23 mL) and water (70 mL) under controlled stirring and maintaining RT. To the workup mixture conc. HCl was added to adjust the pH at 7.0. It was then filtered through celite and washed with ethyl acetate. Combined filtrate was dried (Na₂SO₄) and condensed. Obtained residue was chromatographed (ethyl acetate/hexanes) to obtain 3-(4-hydroxyphenyl)propanol (17 g, 100%) as white solid.

Mp: 52-54° C.

¹H NMR (CDCl₃, 200 MHz δ: 1.78-1.86 (m, 2H); 2.63 (t, J=7.9 Hz, 2H); 3.67 (t, J=6.3 Hz, 2H); 6.74 (d, J=8.8 Hz, 2H); 7.05 (d, J=8.8 Hz, 2H).

IR (neat) cm⁻¹: 3485, 3029, 2940, 1505.

Mass m/z (CI): 152 [M+1].

Step (ii)

To a DCM (550 mL) solution of 3-(4-hydroxyphenyl)propanol (17 g, 1.0 eq, 111.8 mmol), obtained in the step (i) and triethylamine (93.3 mL, 6.0 eq, 670.8 mmol) was added methanesulfonyl chloride (26 mL, 3.0 eq, 335.4 mmol) dropwise at 0° C. The reaction mixture was stirred at RT for 16 h, after that it was worked up by diluting with excess DCM and washing the organic layer with dil. HCl, water and brine. The organic layer was dried (Na₂SO₄) and concentrated. Desired product from the crude mass was purified by recrystallization from diisopropylether. The remaining mother liquor was condensed and was chromatographed (ethyl acetate/hexanes) to obtain further amount desired compound (total yield 20.8 g, 61%) as white solid.

Mp: 60-62° C.

¹H NMR (CDCl₃, 200 MHz: δ 2.00-2.18 (m, 2H); 2.77 (t, J=7.8 Hz, 2H); 3.00 (s, 3H); 3.13 (s, 3H); 4.23 (t, J=6.3 Hz, 2H); 7.22 (aromatics, 4H).

IR (neat) cm⁻¹: 3029, 2935, 1504.

Mass m/z (CI): 309 [M+1].

Step (iii)

### Preparation of (S)-2-Hydroxy-3-(4-nitrophenyl)propionic acid

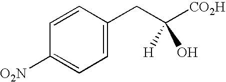

To a solution of (S)-(4-nitrophenyl) glycine (10 g, 47.6 mmol) in a mixture of water (50 mL), H₂SO₄ (1M, 60 mL) and acetone (150 mL) at −5° C., was added under stirring, a solution of sodium nitrite (9.85 g, 142.8 mmol) in water (40 mL) drop wise over a period of 30 min. The reaction mixture was stirred at −5 to 0° C. for another 1.5 h, followed by stirring at room temperature for 16 h. Acetone was removed and then the reaction mixture was diluted with 500 mL ethyl acetate. Organic layer was washed with brine, dried over anhydrous Na₂SO₄, and concentrated. The crude mass was purified by crystallization from isopropyl acetate (9.0 g, 96%).

Mp: 134-136° C.

[α]_{D}: −25° (c 1.0, MeOH)

¹H NMR (CDCl₃) δ: 3.04 (dd, J=14, 7.8 Hz, 1H), 3.24 (dd, J=14.4, Hz, 1H), 4.39 (dd, J=7.3, 4.1 Hz, 1H), 7.42 (d, J=8.7 Hz, 2H), 8.16 (d, J=8.7 Hz, 2H).

IR (neat) cm⁻1: 3485, 3180, 2927, 1715, 1515, 1343.

Mass m/z (CI): 212 (M+1).

Step (iv)

### Preparation of (S)-ethyl-2-hydroxy-3-(4-nitrophenyl) propionate

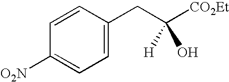

(S)-2-Hydroxy-3-(4-nitrophenyl) propionic acid (9.0 g, 42.6 mmol), obtained from step (iii) above, was dissolved in dry EtOH (300 mL). To this solution was added conc. H₂SO₄ (326 μL, 5.9 mmol), and refluxed for 5 to 6 h. The reaction mixture was neutralized with aqueous sodium bicarbonate. Ethanol was condensed on rotavapor, and the residue was dissolved in ethyl acetate. Organic layer was washed with aqueous sodium bicarbonate, water, brine, and then dried over anhydrous Na₂SO₄, and concentrated. Desired product was obtained from the crude mass by crystallizing from diisopropylether (8.0 g, 78.5%).

Mp: 74-76° C.

[α]_{D}: −13° (c 1.0, MeOH)

¹H NMR (CDCl₃) 1.30 (t, J=7 Hz, 3H), 3.06 (dd, J=14, 7, Hz, 1H), 3.25 (dd, J=14, 4.3, Hz, 1H), 4.25 (q, J=7 Hz, 2H), 4.25 (dd, J=7, 4.3 Hz, 1H), 7.42 (d, J=8.7 Hz, 2H), 8.16 (d, J=8.7 Hz, 2H).

IR (neat) cm⁻¹: 3432, 2924, 1736, 1518, 1347.

Mass m/z (CI): 240 (M+1).

Step (v)

### Preparation of (S)-ethyl-2-methoxy-3-(4-nitrophenyl)propionate

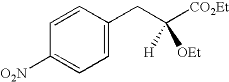

To a mixture of (S)-ethyl-2-hydroxy-3-(4-nitrophenyl)propionate (12.5 g, 52.3 mmol), obtained in step (iv) above, and powdered Ag₂O (36.3 g, 157 mmol) in dry acetonitrile (260 mL) was added methyl iodide (13 mL, 209.2 mmol) at room temperature. Activated molecular sieves (4 A) (12.5 g) were added and then the reaction mixture was stirred at room temperature for 16 h. The reaction mixture was filtered through celite, and concentrated. The crude mass was chromatographed using ethyl acetate and hexanes to obtain the desired product as viscous liquid (10.0 g, 75%).

[α]_{D}: −30.1° (c 1.0, MeOH)

¹H NMR (CDCl₃) δ: 1.24 (t, J=7.1 Hz, 3H); 3.09 (d, J=5.4 Hz, 1H); 3.12 (d, J=2.7 Hz, 1H); 3.35 (s, 3H); 3.96 (dd, J=7.5, 5.1 Hz, 1H); 4.19 (q, J=7.1 Hz, 2H); 7.39 (d, J=8.6 Hz, 2H); 8.13 (d, J=8.6 Hz, 2H).

IR (neat) cm⁻¹: 2995, 1747, 1604, 1521, 1343.

Mass m/z (CI): 254 (M+1).

Step (vi)

### Preparation of (S)-ethyl 2-methoxy-3-(4-aminophenyl)propionate

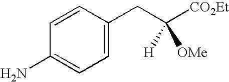

(S)-Ethyl 2-methoxy-3-(4-nitrophenyl)propionate (8.0, 31.6 mmol), obtained in step (v) above, was dissolved in dry methanol (200 mL). To this solution was added 10% Pd/C (2.5 g), and hydrogenated using hydrogen gas (20 psi) for 3-4 h. The reaction mixture was filtered through celite, and concentrated to a syrupy mass. After column chromatography using ethyl acetate/hexanes the desired product was isolated as thick liquid (7.0 g, quantitative).

[α]_{D}: −14.1°(c 1.0, MeOH).

Chiral HPLC: >98% ee.

¹H NMR (CDCl₃) δ: 1.23 (t, J=7.2 Hz, 3H), 2.91 (d, J=6.1 Hz, 2H), 3.30 (bs, 2H, NH₂), 3.34 (s, 3H), 3.88 (t, J=6.2 Hz, 1H), 4.17 (q, J=7.2 Hz, 2H), 6.62 (d, J=8.3 Hz, 2H), 7.01 (d, J=8.1 Hz, 2H).

IR (neat) cm⁻¹: 3372, 2985, 2932, 1739, 1627, 1519.

Mass m/z (CI): 223 (M), 234 (M+1), 192 (M-OMe).

Step (vii)

### Preparation of (S) Ethyl 2-methoxy-3-[4-{3-(4-methanesulfonyloxy phenyl) propylamino} phenyl] propionate

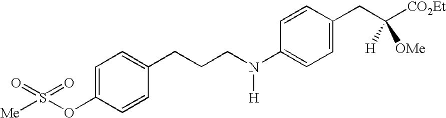

A mixture of 4-(3-methanesulfonyloxypropyl)phenylmethanesulfonate (5.5 g, 1.0 eq, 17.9 mmol), obtained in step (ii), (S) ethyl 2-methoxy-3-(4-aminophenyl) propionate (4.0 g, 1.0 eq, 17.9 mmol), obtained in step (vi), tetrabutylammonium bromide (2.8 g, 0.5 eq, 9.0 mmol) and anhydrous K₂CO₃ (7.4 g, 3.0 eq, 53.7 mmol) in dry toluene (90 mL) was heated with stirring at 90° C. for 7-9 h. The reaction mixture was diluted with ethyl acetate (300 mL) and washed with water and brine. Organic layer was dried (Na₂SO₄), condensed, and the residue was chromatographed using ethyl acetate and hexanes to obtain the title compound as viscous liquid (3.4 g, 44%).

[α]25_{D}: −6.5° (c 1.0, MeOH).

¹H NMR (CDCl₃, 200 MHz): δ 1.26 (t, J=7.0 Hz, 3H); 1.98 (quintet, J=7.2 Hz, 2H); 2.75 (t, J=7.6 Hz, 2H); 2.93 (d, J=5.9 Hz, 2H); 3.02-3.22 (m, 5H); 3.37 (s, 3H); 3.91 (t, J=6.4 Hz, 1H); 4.20 (q, J=7.0 Hz, 2H); 6.65 (d, J=8.0 Hz, 2H); 7.08 (d, J=8.3 Hz, 2H); 7.15-7.3 (aromatics, 4H).

IR (neat) cm⁻¹: 3405, 2934, 2934, 1739, 1617, 1522, 1367.

Mass m/z (CI): 435 [M], 436 [M+1].

Step (viii)

### Preparation of (S) 3-Methoxy-3-[4-{3-(4-methanesulfonyoxy phenyl)propylamino} phenyl]propionic acid]

(S) Ethyl 2-methoxy-3-[4-{3-(4-methanesulfonyloxyphenyl) propylamino}phenyl] propionate (3.4 g, 1.0 eq, 7.8 mmol), obtained in step (vii) above, was hydrolyzed by treating with LiOH.H₂O (492 mg, 1.5 eq, 11.7 mmol) in MeOH-THF-water solvent mixture at RT for 3-4 h. The reaction mixture was condensed, diluted with water and acidified (pH at 3) with aq. HCl. Desired acid was precipitated out from aqueous layer, which was then filtered out. If the precipitated acid was not pure enough by TLC, it was chromatographed using MeOH and CHCl₃ as eluents to obtain the pure acid as white solid (2.5 g, 79%).

Mp: 90-92° C.

[α]d: −16° (c 1.0, MeOH).

¹H NMR (CDCl₃, 200 MHz): δ 1.25 (s, 1H, N—H); 1.94 (quintet, 7.2 Hz, 2H); 2.72 (t, J=7.8 Hz, 2H); 2.82-3.02 (m, 2H); 3.02-3.18 (m, 5H); 3.38 (s, 3H); 3.97 (t, J=4.8 Hz, 1H); 4.90 (bs, CO₂H)); 6.58 (d, J=8.1 Hz, 2H); 7.05 (d, J=8.3 Hz, 2H); 7.15-7.24 (aromatics, 4H).

¹³C NMR (CDCl₃, 50 MHz) δ: 29.53, 32.34, 37.15 (2C), 46.48, 57.58, 82.07, 116.31, 121.90, 129.75, 130.39, 140.57, 142.48, 147.33, 175.87.

IR (neat) cm⁻¹: 3046, 2932, 1732, 1615, 1520, 1365.

Mass m/z (CI): 408 [M+1], 407 [M].

See also Example 14 of WO 03/048116, which is hereby incorporated by reference as part of Example 1.

### EXAMPLE 2

### L-Arginine salt of (s)-2-methoxy-3-[4-{3-(4-Methanesulfonyloxyphenyl) propylamino} phenyl]propionicacid in monohydrated form (Arginine salt monohydrate)

To a mixture of (S)-2-Methoxy-3-[4-{3-(4-methanesulfonyloxyphenyl) propylamino} phenyl] propionicacid (10 g) and DM water (30 ml), L-Arginine (0.426 g) were refluxed added to the reaction mixture at 25-30° C. in about 5 minutes under stirring and maintained the reaction mixture at 50-70° C. for 4-5 hours. Isopropanol (120 mL) was added to the reaction mixture at same temperature and heated to reflux temperature of the solvent, and maintained for 1-2 hours to get clear solution. Then cooled to 25-35° C. in about 5-6 hours and maintained for 24 hours under stirring at 25-30° C. The precipitated product was filtered, dried at 60° C. for 8-10 hours to afford pure L-Arginine salt of (S)-2-methoxy-3-[4-{3-(4-Methanesulfonyloxyphenyl)propylamino} phenyl] propionic acid in monohydrated form, as off white crystalline solid. (10 g, 90%)

Melting point: 125-135° C.

Purity: 98.15% by HPLC.

Mass: 408 (M+1).

The infrared spectrum of a KBr pellet of the product was obtained using a FT-IR spectrometer at 4 cm⁻¹ resolution (FIG. 1). Bands were observed at 3386, 1630, 1364, 1152, 971, 871.

The X-Ray Powder Diffractogram pattern of the product (FIG. 2) was recorded using the following acquisition conditions: Tube anode: Cu, Generator tension: 50 kV, Generator current: 34 mA, Start angle: 3.0°2θ, End angle: 45°2θ, Step size: 0.02°2θ, speed: 3 deg./min. Characteristic XRPD angles and relative intensities are recorded in Table 1.

TABLE 1

| Angle | |
|---|---|
| 2-Theta ° | Relative intensity |
| 7.20 | 11 |
| 8.44 | 43 |
| 12.68 | 20 |
| 14.22 | 9 |
| 14.80 | 8 |
| 15.96 | 32 |
| 17.02 | 11 |
| 17.48 | 49 |
| 17.60 | 68 |
| 18.02 | 11 |
| 18.84 | 40 |
| 19.22 | 9 |
| 20.00 | 6 |
| 21.02 | 100 |
| 21.18 | 56 |
| 21.66 | 38 |
| 22.02 | 78 |
| 22.38 | 23 |
| 22.98 | 23 |
| 23.14 | 17 |
| 23.80 | 18 |
| 24.40 | 28 |
| 26.36 | 11 |
| 26.92 | 14 |
| 28.68 | 6 |
| 29.12 | 17 |
| 29.68 | 7 |
| 29.96 | 15 |
| 33.56 | 9 |
| 35.74 | 7 |
| 36.20 | 7 |
| 36.56 | 8 |

Tₒₙₛₑₜ of ‘Arginine hydrate’: The Tₒₙₛₑₜ was determined by Differential Scanning Calorimetry. Melting endotherm was observed at 92.6° C., 132.53° C., 231.80° C., 272.22° C.

### EXAMPLE 3

### L-Arginine salt of (S)-2-methoxy-3-[4-{3-(4-methanesulfonyloxyphenyl) propylamino} phenyl} propionicacid (Arginine salt)

(S)-2-Methoxy-3-[4-{3-(4-methanesulfonyloxyphenyl)propylamino} phenyl} propionicacid, (10 grams) and DM water (30 mL) were refluxed. L-Arginine (0.426 grams) was added to the reaction mixture at 25-30° C. in about 5 minutes and maintained at the same temperature for 4-5 hours. Isopropanol (120 mL) was added to the reaction mixture and continued stirring further for 2 to 4 hours. The precipitated product was filtered, dried at 60° C. for 8-10 hours and further refluxed with toluene to remove water azeotropically. Reaction mass cooled to 25-30° C. and filtered and washed with toluene, dried at 60-65° C. for 8-10 hours to afford the pure form of L-Arginine salt of (s)-2-methoxy-3-[4-{3-(4-methanesulfonyloxyphenyl) propylamino}phenyl} propionicacid, as off white crystalline solid. (10 grams, 90%)

Melting point 190-194° C.

Purity: 98.15% by HPLC.

Mass: 408 (M+1).

The infrared spectrum of a KBr pellet of the product was obtained using a FT-IR spectrometer at 4 cm⁻¹ resolution (FIG. 4). The X-Ray Powder Diffractogram pattern of the product (FIG. 5) was recorded using the following acquisition conditions: Tube anode: Cu, Generator tension: 50 kV, Generator current: 34 mA, Start angle: 3.0°2θ, End angle: 45°2θ, Step size: 0.02°2θ, speed: 3 deg./min. Characteristic XRPD angles and relative intensities are recorded in Table 2.

TABLE 2

| Angle | |
|---|---|
| 2-Theta ° | Relative intensity |
| 4.06 | 8 |
| 8.10 | 9 |
| 11.72 | 9 |
| 12.10 | 16 |
| 12.62 | 18 |
| 13.66 | 14 |
| 14.06 | 12 |
| 15.16 | 8 |
| 15.28 | 7 |
| 16.40 | 56 |
| 16.80 | 100 |
| 17.38 | 68 |
| 18.02 | 10 |
| 18.64 | 28 |
| 19.28 | 56 |
| 19.70 | 70 |
| 20.22 | 96 |
| 20.56 | 42 |
| 20.92 | 37 |
| 21.30 | 32 |
| 22.10 | 13 |
| 22.56 | 12 |
| 22.86 | 18 |
| 23.50 | 21 |
| 23.66 | 30 |
| 24.10 | 38 |
| 25.28 | 23 |
| 25.82 | 6 |
| 26.02 | 8 |
| 26.42 | 6 |
| 27.70 | 8 |
| 28.34 | 9 |
| 29.34 | 6 |
| 29.88 | 7 |
| 30.34 | 9 |
| 30.74 | 6 |
| 30.96 | 10 |
| 31.32 | 7 |
| 31.68 | 6 |
| 32.96 | 7 |
| 33.14 | 10 |
| 36.16 | 6 |

Tₒₙₛₑₜ of ‘Arginine hydrate’: The Tₒₙₛₑₜ was determined by Differential Scanning Calorimetry. Melting endotherm was observed at 192.93° C., 228.69° C., 272.22° C.

### EXAMPLE 4

### L-Arginine salt of (S)-2-methoxy-3-[4-{3-(4-methanesulfonyloxyphenyl) propylamino}phenyl]propionic acid (conversion from Arginine salt monohydrate to Arginine salt)

L-Arginine salt of (S)-2-Methoxy-3-[4-{3-(4-methanesulfonyloxyphenyl) propylamino}phenyl] propionic acid in monohydrated form (Arginine monohydrate) (10 grams) and toluene (100 mL) was refluxed for 3-4 hours to remove water azeotropically. Reaction mass cooled to 25-30° C. and filtered and washed with toluene, dried at 60-65° C. for 8-10 hours to afford the pure form of L-Arginine salt of (S)-2-methoxy-3-[4-{3-(4-methanesulfonyloxyphenyl)propylamino} phenyl]propionic acid, as off white crystalline solid. (10 grams, 90%)

Melting point.: 190-194° C.

Purity: 98.15% by HPLC.

Mass: 408 (M⁺+1).

X-ray diffraction (I/I₀): 16.920 (100), 20.340 (90), 20.200 (83), 17.480 (72) 19.800 (69), 19.680 (60), 19.360 (55), 21.080 (55), 20.700 (40) 24.160 (38).

### EXAMPLE 5

### L-Arginine salt of (S)-2-methoxy-3-[4-{3-(4-methanesulfonyloxyphenyl) propylamino}phenyl] propionic acid in monohydrated form (conversion from Arginine salt to Arginine salt monohydrate)

L-Arginine salt of (S)-2-Methoxy-3-[4-{3-(4-methanesulfonyloxyphenyl) propylamino}phenyl] propionic acid (10 grams) and water (40 mL) were heated at 50-60° C. for 4-5 hours. Isopropanol (120 mL) was added to the reaction mixture at same temperature and heated to reflux temperature and maintained for 1-2 hours to get clear solution. Then cooled to 25-35° C. in about 5-6 hours and maintained for 2-4 hours under stirring at 25-30° C. The precipitated product was filtered, dried at 60° C. for 8-10 hours to afford pure L-Arginine salt of (S)-2-methoxy-3-[4-{3-(4-methanesulfonyloxyphenyl) propylamino}phenyl}propionic acid in monohydrate form, as off white crystalline solid. (10 grams, 90%)

Melting point: 125-135° C.

Purity: 98.15% by HPLC.

Mass: 408 (M⁺+1).

X-ray diffraction (I/I₀): 22.02 (100), 21.02 (98), 8.440 and 17.600 (64), 21.2 (60), 17.480 (46), 21.640 (40).

### EXAMPLE 6

a) Determination of hPPARα Activity

Ligand binding domain of hPPARα was fused to DNA binding domain of Yeast transcription factor Gal 4 in eucaryotic expression vector. Using superfect (Qiagen, Germany) as transfecting reagent HEK-293 cells are transfected with this plasmid and a reporter plasmid harboring the luciferase gene driven by a GAL4 specific promoter. Compound can be added at different concentrations after 42 hrs of transfection and incubated overnight. Luciferase activity as a function of compound binding/activation capacity of PPARα will be measured using Packard Luclite kit (Packard, USA) in Top Count (Ivan Sadowski, Brendan Bell, Peter Broag and Melvyn Hollis. Gene. 1992. 118: 137-141; Superfect Transfection Reagent Handbook. February 1997. Qiagen, Germany).

b) Determination of hPPARγ Activity

Ligand binding domain of hPPARγ1 is fused to DNA binding domain of Yeast transcription factor GAL4 in eucaryotic expression vector. Using lipofectamine (Gibco BRL, USA) as transfecting reagent HEK-293 cells are transfected with this plasmid and a reporter plasmid harboring the luciferase gene driven by a GAL4 specific promoter. Compound can be added at 1 μM concentration after 48 hrs of transfection and incubated overnight. Luciferase activity as a function of drug binding/activation capacity of PPARγ1 will be measured using Packard Luclite kit (Packard, USA) in Packard Top Count (Ivan Sadowski, Brendan Bell, Peter Broag and Melvyn Hollis. Gene. 1992. 118: 137-141; Guide to Eukaryotic Transfections with Cationic Lipid Reagents. Life Technologies, GIBCO BRL, USA).

c) Determination of HMG CoA Reductase Inhibition Activity

Liver microsome bound reductase is prepared from 2% cholestyramine fed rats at mid-dark cycle. Spectrophotometric assays are carried out in 100 mM KH₂PO₄, 4 mM DTT, 0.2 mM NADPH, 0.3 mM HMG CoA and 125 μg of liver microsomal enzyme. Total reaction mixture volume was kept as 1 ml. Reaction was started by addition of HMG CoA. Reaction mixture is incubated at 37° C. for 30 min and decrease in absorbance at 340 nm was recorded. Reaction mixture without substrate was used as blank (Goldstein, J. L and Brown, M. S. Progress in understanding the LDL receptor and HMG CoA reductase, two membrane proteins that regulate the plasma cholesterol. J. Lipid Res. 1984, 25: 1450-1461). The test compounds will inhibit the HMG CoA reductase enzyme.

Unless stated to the contrary, words and phrases such as “including,” “containing,” “comprising,” “having”, “for example”, “i.e.”, “in particular” and the like, means “including without limitation” and shall not be construed to limit any general statement that it follows to the specific or similar items or matters immediately following it. Except where the context indicates to the contrary, all exemplary values are intended to be used for purposes of illustration. Most of the foregoing alternative embodiments are not mutually exclusive, but may be implemented in various combinations. As these and other variations and combinations of the features discussed above can be utilized without departing from the invention as defined by the claims, the foregoing description of the embodiments should be taken by way of illustration rather than by way of limitation of the invention as defined by the appended claims.

1. Arginine salt of (S)-2-methoxy-3-[4-{3-(4-methanesulfonyloxyphenyl) propylamino}phenyl] propionic acid monohydrate.

2. Arginine salt of (S)-2-methoxy-3-[4-{3-(4-methanesulfonyloxyphenyl) propylamino}phenyl] propionic acid.

3. (S)-2-methoxy-3-[4-{3-(4-methanesulfonyloxyphenyl)propylamino} phenyl] propionic acid monohydrate.

4. A basic salt of (S)-2-methoxy-3-[4-{3-(4-methanesulfonyloxyphenyl) propylamino}phenyl] propionic acid monohydrate.

5. An amino acid salt of (S)-2-methoxy-3-[4-{3-(4-methanesulfonyloxyphenyl)propylamino}phenyl] propionic acid monohydrate.

6. A basic salt of (S)-2-methoxy-3-[4-{3-(4-methanesulfonyloxyphenyl) propylamino}phenyl] propionic acid.

7. An amino acid salt of (S)-2-methoxy-3-[4-{3-(4-methanesulfonyloxyphenyl)propylamino}phenyl] propionic acid.

8. A pharmaceutical composition comprising:
an API which is a basic salt of (S)-2-methoxy-3-[4-{3-(4-methanesulfonyloxyphenyl) propylamino}phenyl]propionic acid, a basic salt of (S)-2-methoxy-3-[4-{3-(4-methanesulfonyloxyphenyl) propylamino}phenyl]propionic acid monohydrate or (S)-2-methoxy-3-[4-{3-(4-methanesulfonyloxyphenyl)propylamino} phenyl]propionic acid monohydrate and a pharmaceutically acceptable carrier, diluent, excipient or solvent.

9. The composition of claim 8, wherein said API is in crystalline form.

10. The composition of claim 8, wherein said API is present in an amount of at least 0.10% by weight of the composition.

11. The composition of claim 10, wherein said API is present in an amount of at between about 0.10 and about 12.0 mg.

12. The composition of claim 9, wherein said API is present in an amount of at least 0.10% by weight of the composition.

13. The composition of claim 12, wherein said API is present in an amount of at between about 0.10 and about 12.0 mg.

14. A solid oral dosage form comprising:
an API which is a basic salt of (S)-2-methoxy-3-[4-{3-(4-methanesulfonyloxyphenyl) propylamino}phenyl]propionic acid, a basic salt of (S)-2-methoxy-3-[4-{3-(4-methanesulfonyloxyphenyl) propylamino}phenyl]propionic acid monohydrate or (S)-2-methoxy-3-[4-{3-(4-methanesulfonyloxyphenyl) propylamino}phenyl]propionic acid monohydrate, at least one filler and at least one lubricant, said dosage form being a powder, granule, agglomerate, lozenge, pastille, reconstitutable powder, tablet, capsule, caplet or gum.

15. The solid oral dosage form of claim 14, wherein said API is in crystalline form.

16. The solid oral dosage form of claim 14, wherein said API is present in an amount of at least 0.10% by weight of the composition.

17. The solid oral dosage form of claim 14, wherein said API is present in an amount of at between about 0.10 and about 12.0 mg.

18. The solid oral dosage form of claim 15, wherein said API is present in an amount of at least 0.10% by weight of the composition.

19. The solid oral dosage form of claim 18, wherein said API is present in an amount of at between about 0.10 and about 12.0 mg.
